(19) United States
(12) Patent Application Publication
(10) Pub. No.: US 2026/0026827 A1
(43) Pub. Date: Jan. 29, 2026

(54) GUIDEWIRE AND MICROCATHETER

(71) Applicant: ENDOWAYS (HONG KONG) MEDICAL CO., LIMITED, Hong Kong (HK)

(72) Inventors: Yaniv KIRMA, Pardes Hana - Karkur (IL); German ALEYNIKOV, Zikhron Ya'akov (IL); Amos BRACHA, Hadera (IL)

(21) Appl. No.: 18/997,087

(22) PCT Filed: Jul. 18, 2023
(86) PCT No.: PCT/IL2023/050749

§ 371 Date: Jan. 19, 2025

Related U.S. Application Data
(60) Provisional application No. 63/390,361, filed on Jul. 19, 2022.
(60) Provisional application No. 63/391,752, filed on Jul. 24, 2022.

(51) Int. Cl. A61B 17/221; A61B 17/00; A61B 17/22; A61B 90/00; A61M 25/00; A61M 25/09

(52) U.S. Cl. CPC A61B 17/221; A61B 90/39; A61M 25/0021; A61M 25/0043; A61M 25/09; A61B 2017/00367; A61B 2017/22042; A61B 2017/2212; A61B 2090/3966; A61M 2025/0042; A61M 2025/09083; A61M 2025/09133; A61M 2025/09166; A61M 2025/09175

(57) ABSTRACT

A medical guide wire for improved blood vessel navigation (i.e., to a blood clot, aneurism or hemorrhagic stroke) includes a wire having a curved distal tip, a shaft covering at last part of the wire, and a coil affixedly covering a portion of the wire proximal to the distal tip and distal to the shaft. Moving the shaft linearly toward the coil compresses the coil and rotates the distal tip. A microcatheter includes a tube having a braided distal portion that is foldable axially. The braided distal portion may comprise a mixture of thicker and thinner strands. When a mechanism triggers the folding, the braided portion's outer diameter increases. The mechanism may be an actuator on the guide wire, which when pulled, exerts axial pressure (directly or indirectly) against a braided portion's distal end. A blood clot nearby may be caught and the microcatheter removed with the blood clot.

### FIELD OF THE INVENTION

The invention is in the field of medical guidewires and microcatheters and combinations of such guide wires and microcatheters.

### BACKGROUND OF THE INVENTION

Doctors may need to navigate a blood vessel in the brain for several reasons. One reason to reach and remove a blood clot that is ischemic. Another is to reach and treat an area of a hemorrhagic stroke. A third reason is to reach and treat an aneurism in which a bulge forms in a blood vessel caused by a weakness in the blood vessel wall, usually where it branches.

There is a limited amount of time from when a blood clot, for example an ischemic (stroke) blood clot forms until its removal is clinically effective. Clot removal is the standard of care with acute stroke if it is within the first six hours after symptom onset. From 6 hours to 24 hours the evidence is less clear if this is the best course of treatment.

Guided by real-time imaging of the blood vessels, a surgeon must navigate a catheter within the blood vessels up to the arteries in the brain and then carefully removes the clot using a variety of devices, including suction and/or a stent-like device. This procedure takes time and typically it does not even begin until about three hours after the patient discovers symptoms.

When a patient with symptoms comes to the emergency room it takes a certain of time to gather information, run a CT image, inject the patient with an imaging contrast material, interpret the images to discover the problem and turn the patient over to the surgeon. That tends to consume about 3 hours. The surgeon therefore has a limited amount of time to reach the blood vessel to perform a clinically effective intervention.

### SUMMARY OF THE EMBODIMENTS

Applicant has determined that many blood vessels in which a blood clot or aneurism or hemorrhagic stroke has to be reached by the surgeon are difficult to reach because the blood vessel may be bent, looped or bifurcated. As a result, the process of the surgeon navigating to the desired region of the blood vessel where the clinical intervention is to take place is arduous. Accordingly, the surgeon often spends a lot of time, sometimes hours, navigating the blood vessel with the guide wire, catheter or microcatheter just to get to the area or location of the problem in the blood vessels.

One embodiment is a medical guide wire for improved blood vessel navigation, comprising a wire having a curved distal tip; a shaft covering at last part of the wire; a coil affixedly covering a portion of the wire proximal to the distal tip and distal to the shaft, wherein moving the shaft linearly toward the coil compresses the coil and rotates the distal tip.

In some embodiments, moving the shaft linearly away from the coil releases the coil and rotates the distal tip in an opposite direction.

In some embodiments, compressing the coil rotates the distal tip up to 720 rotational degrees.

In some embodiments, compressing the coil rotates the distal tip from 90 to 720 rotational degrees.

In some embodiments, the distal tip is radiopaque.

In some embodiments, a radius of the distal tip is about 3 mm.

In some embodiments, the guide wire further comprises a tube inside the shaft.

In some embodiments, the coil is made of a metal alloy of nickel and titanium.

In some embodiments, the guide wire is in combination with a microcatheter.

In some embodiments, the wire is substantially straight other than the curved distal tip.

Another embodiment is a microcatheter and guide wire combination, comprising the microcatheter comprising a tube having a braided distal portion that is foldable axially; the guide wire inside the microcatheter and comprising a wire having at a distal end a mechanism configured to actuate an axial folding of the braided distal portion of the microcatheter.

In some embodiments, the braided distal portion in an unfolded position has a first outer diameter and in a folded position has a second outer diameter such that the second outer diameter is larger than the first outer diameter.

In some embodiments, the braided distal portion in an unfolded position has a maximum first outer diameter and in a folded position has a maximum second outer diameter such that the maximum second outer diameter is larger than the maximum first outer diameter. In some embodiments, the maximum second outer diameter is at least twice the maximum first outer diameter.

In some embodiments, the braided distal portion in an unfolded position has an average first outer diameter and in a folded position has a maximum second outer diameter such that the maximum second outer diameter is at least twice the average first outer diameter.

In some embodiments, the mechanism comprises a flange, bump or annular bulge such that pulling the guide wire proximally induces the flange, bump or annular bulge to press against a distal end of (i) the braided distal portion or (ii) a portion distal to the braided distal portion.

In some embodiments, the braided distal portion comprises a mixture of thicker and thinner strands.

In some embodiments, the braided distal portion comprises a symmetrical mixture of thicker and thinner strands.

In some embodiments, the braided distal portion comprises a first braided portion and a second braided portion separated by a non-braided portion.

In some embodiments, the wire has a curved distal tip, the guide wire also including a shaft covering at least part of the wire and a coil affixedly covering a portion of the wire proximal to the distal tip and distal to the shaft, wherein moving the shaft linearly toward the coil compresses the coil and rotates the distal tip.

In some embodiments, the braided distal portion includes a metal alloy of nickel and titanium.

In some embodiments, the braided distal portion is configured to catch a blood clot alongside the braided distal portion when the microcatheter is in a blood vessel.

In some embodiments, the guide wire is substantially straight other than at a curved distal tip of the guide wire.

Another embodiment is a microcatheter comprising a tube having a braided distal portion that is axially foldable.

In some embodiments, the braided distal portion comprises a mixture of thicker and thinner strands.

In some embodiments, the braided distal portion comprises a first braided portion and a second braided portion separated by a non-braided portion.

In some embodiments, the braided distal portion is configured to catch a blood clot alongside the braided distal portion when the microcatheter is in a blood vessel.

In some embodiments, the braided distal portion in an unfolded position has a first outer diameter and in a folded position has a second outer diameter such that the second outer diameter is larger than the first outer diameter.

In some embodiments, the braided distal portion in an unfolded position has a maximum first outer diameter and in a folded position has a maximum second outer diameter such that the maximum second outer diameter is larger than the maximum first outer diameter. In some embodiments, the maximum second outer diameter is at least twice the maximum first outer diameter.

In some embodiments, the braided distal portion in an unfolded position has an average first outer diameter and in a folded position has a maximum second outer diameter such that the maximum second outer diameter is at least twice the average first outer diameter.

In some embodiments, the braided distal portion includes a metal alloy of nickel and titanium.

Another embodiment is a method of inserting a guide wire into a blood vessel that contains a difficult-to-navigate portion, the method comprising inserting the guide wire into the blood vessel, wherein the guide wire including a wire having a curved distal tip, and including a shaft covering part of the wire and a coil affixedly covering a portion of the wire proximal to the distal tip and distal to the shaft; navigating a bent, looped or bifurcated portion of the blood vessel by moving the shaft linearly toward the coil so as to compress the coil, the compression rotating the distal tip, so as to reach one of a blood clot, an aneurism and a hemorrhagic stroke.

Another embodiments is a method of removing a blood clot from a blood vessel of a mammalian subject, comprising inserting a microcatheter into the blood vessel over a guide wire, the microcatheter having an axially foldable braided distal portion, such that a distal end of the guide wire extends further than a distal end of the microcatheter; and pulling the guide wire proximally so that a mechanism on the guide wire triggers an axial folding of the braided distal portion.

In some embodiments, the method further comprises axially folding the braided distal portion so as to catch a blood clot external to the microcatheter. In some embodiments, the method further comprises withdrawing the catheter with the blood clot.

In some embodiments, the mechanism comprises a flange, bump or annular bulge that presses against a distal end of (i) the braided distal portion or of (ii) a portion distal to the braided distal portion.

In some embodiments, the guide wire comprises a wire having a curved distal tip, a shaft covering part of the wire and a coil affixedly covering a portion of the wire proximal to the distal tip and distal to the shaft such that linear movement of the shaft compresses the coil and rotates the distal tip.

Another embodiment is a method of treating an aneurism, comprising inserting the guide wire into the blood vessel, the guide wire including a wire having a curved distal tip, and including a shaft covering part of the wire and a coil affixedly covering a portion of the wire proximal to the distal tip and distal to the shaft; navigating a bent, looped or bifurcated portion of the blood vessel to reach the aneurism by moving the shaft linearly toward the coil so as to compress the coil, the compression rotating the distal tip; and inserting a microcatheter and treating the aneurism using one of (i) endovascular coiling, (ii) a covered stent and (iii) a biological graft.

Another embodiment is a system for navigating difficult to navigate blood vessels, comprising a robot including at least one engine and an actuator; an input device; a microcatheter and guide wire combination, comprising: the microcatheter comprising a tube having a braided distal portion that is foldable axially; the guide wire inside the microcatheter and comprising a wire having at a distal end a mechanism configured to actuate an axial folding of the braided distal portion of the microcatheter.

### BRIEF DESCRIPTION OF THE DRAWINGS

Various embodiments are herein described, by way of example only, with reference to the accompanying drawings, wherein:

FIG. 1 is a perspective view of a guide wire, in accordance with one embodiment;

FIG. 2 is a side view of the guide wire showing the shaft in a neutral position positioned for linear movement to compress the coil and rotate the distal tip, in accordance with one embodiment;

FIG. 3 is a side view of the guide wire showing the shaft having compressed the coil and rotated the distal tip and in position for releasing the coil and rotating the distal tip in an opposite direction, in accordance with one embodiment;

FIG. 4 is a side view of a microcatheter showing braided portions is in an unfolded position, in accordance with one embodiment;

FIG. 5 is a side view of the microcatheter of FIG. 4 showing the braided portions in an axially folded position, in accordance with one embodiment;

FIG. 6 is side view of a guide wire showing its detailed components, in accordance with one embodiment;

FIG. 7 is a sectional view taken along line C-C of FIG. 6, in accordance with one embodiment;

FIG. 8 is an enlarged view of detail A of FIG. 6, in accordance with one embodiment;

FIG. 9 is an enlarged view of a coil of the guide wire, in accordance with one embodiment;

FIG. 10 is a flow chart of a method, in accordance with one embodiment;

FIG. 11 is a flow chart of another method, in accordance with one embodiment;

FIG. 12 is a flow chart showing a further method, in accordance with one embodiment; and

FIG. 13 is a schematic drawing of a system, in accordance with one embodiment.

### DETAILED DESCRIPTION OF THE EMBODIMENTS

The following detailed description is of the best currently contemplated modes of carrying out the invention. The description is not to be taken in a limiting sense, but is made merely for the purpose of illustrating the general principles of the invention, since the scope of the invention is best defined by the appended claims.

Certain embodiments generally provide a guide wire for navigating difficult to navigate blood vessels. Certain embodiments provide a microcatheter to be used either with the guide wire or with another guide wire. In certain embodiments, there is a combination of the guide wire and the microcatheter.

The guide wire is for use in navigating difficult to navigate blood vessels, for example looped, bent or bifurcated blood vessels. This navigation may be to reach a blood clot in preparation for removing blood clots, to reach an area of an aneurism or hemorrhagic stroke in preparation for repairing the aneurism or treating the hemorrhagic stroke or another intervention by means of a microcatheter.

It can be an arduous task for the surgeon to situate the guide wire and microcatheter at the point of interest in the blood vessel, for example at a blood clot, aneurism or hemorrhagic stroke. The sooner the surgeon reaches the point of interest and sets up the microcatheter there, the sooner the clinical intervention can be effective. The amount of time that passes before the clinical intervention may directly affect the clinical outcome. The surgeon may inadvertently pass the juncture of a bifurcated blood vessel and need to withdraw and partially re-navigate correctly. Alternatively, the surgeon may reach the desired location only to have it slip away. The surgeon would then need additional maneuvering, which takes up precious time.

One embodiment is a guide wire that includes a wire, for example a substantially straight wire, having a curved distal tip, and a mechanism for converting linear movement to rotational movement of the distal tip. For example, the guide wire may include a shaft covering at last part of the wire and a coil affixedly covering a portion of the wire proximal to the distal tip and distal to the shaft. Instead of having to rotate the tip of the guide wire (or microcatheter) by means of a rotational movement of a more proximal portion of the guide wire, which can be difficult, the shaft of the guide wire is configured to be moved linearly, for example toward the coil. This linear movement is configured to exert an axial compressive force so as to compress the coil, thereby automatically rotating the distal tip. The rotation of the distal tip enables the guide wire to enter difficult to access spaces in the blood vessel. This makes it significantly easier to efficiently navigate difficult to navigate blood vessels, for example those that are bifurcated, looped or bent. Accordingly, a significant amount of time is saved buy the surgeon.

Guide wires are used together with microcatheters and placed before the microcatheter is placed. In this case, in addition to a novel guide wire, a novel microcatheter is also presented, as described below. Accordingly, embodiments of the novel guide wire described herein may be used together with a microcatheter that is not braided or they may be in combination with embodiments of the novel braided microcatheter described herein. Likewise, embodiments of the braided microcatheter described herein may be used together with a guide wire that has the feature of a mechanism for triggering folding of the braids of the microcatheter or that has the features of the linear movement of the shaft causing the rotation of the distal tip or both features (or neither features if there is some other mechanism for triggering the folding of the braids without a mechanism of the guide wire).

The microcatheter may be a braided microcatheter. The microcatheter may include a braided portion. The microcatheter may have a braided distal portion that may be axially foldable when compression is applied upon the triggering of a mechanism. Once the guide wire is in place, the microcatheter may be positioned over the guide wire. In one non-limiting example of the mechanism, the distal end of the guide wire may stick out of the distal end of the microcatheter. By pulling the guide wire back (proximally) a mechanism, for example a mechanism that is on the guide wire, may trigger a compression of the braided portion of the microcatheter. For example, when the guide wire is pulled back in a proximal direction, an actuator on the guide wire may press against a distal end of the braided section of the microcatheter. In one non-limiting implementation of this, a flange, bulge or bump on the distal portion of the guide wire may press against a distal end of the braided section (or against a portion distal to the braided section) of the microcatheter to generate a compressive force that axially folds the braided portion of the microcatheter. The axial folding may cause an increase in outer diameter of all or part of the braided portion of the microcatheter. When the braided portion bulges outward as a result of the axial folding, a nearby blood clot alongside the microcatheter may be snagged and caught in the braided strands of the braided sections of the microcatheter. Consequently, when the microcatheter is then removed from the blood vessel, the blood clot may be “dragged” along with it and automatically removed together with the microcatheter.

In the case of an aneurism or the hemorrhagic stroke, the advantage of the guide wire is to navigate the difficult to navigate blood vessel and reach the desired location namely the aneurism or the hemorrhagic stroke promptly so as to commence treatment.

The entire procedure of using the guide wire and the microcatheter (or each alone) may be guided by a smart robot. In that case, the surgeon is able to control the robot with a joystick or other input device. As a result, the surgeon can distance himself (for example by being in a location separated by a wall from the actual device) from the X-ray or other imaging that may be taken during the procedure using the radiopaque distal end or catheter braids.

The term “about” as used herein refers to plus or minus 10% unless otherwise specified. For example, “about X millimeters” means between 0.9× and 1.1× millimeters.

The term “straight wire” as used herein in any embodiment refers to a guide wire that starts off being substantially straight (other than the portion of said guide wire such as a curved tip that is specifically described as being other than straight) in its natural position, for example before use. Its “natural position” refers to its position when placed fully opened to its maximum length on a flat surface without subjecting it to an external force (other than the ambient air) that stretches or bends it. This of course does not preclude the fact that such substantially straight guide wire may well curve or bend when inserted into a curved or bent blood vessel.

The principles and operation of a Guidewire and Microcatheter may be better understood with reference to the drawings and the accompanying description.

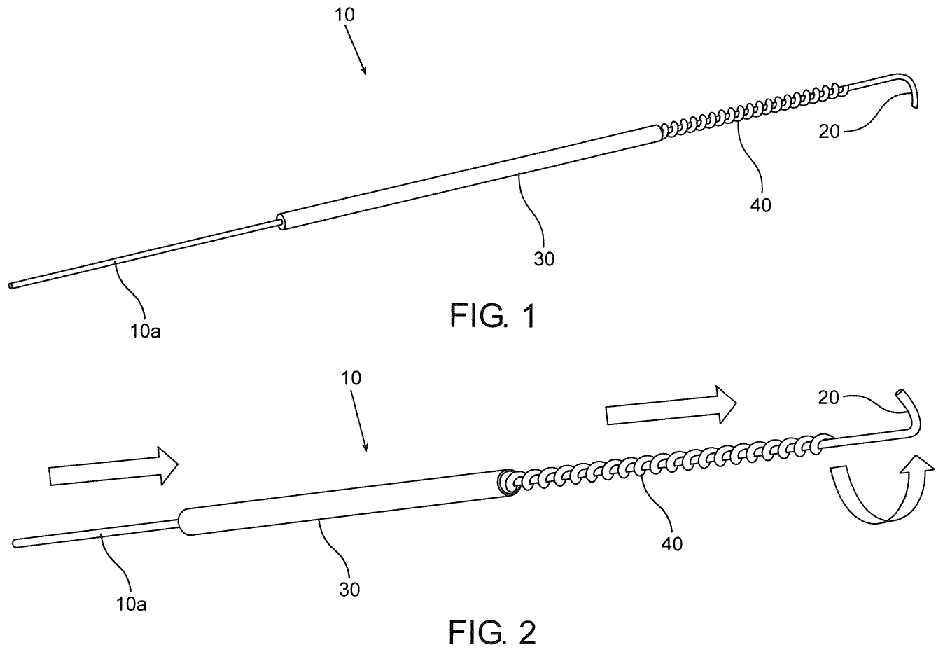

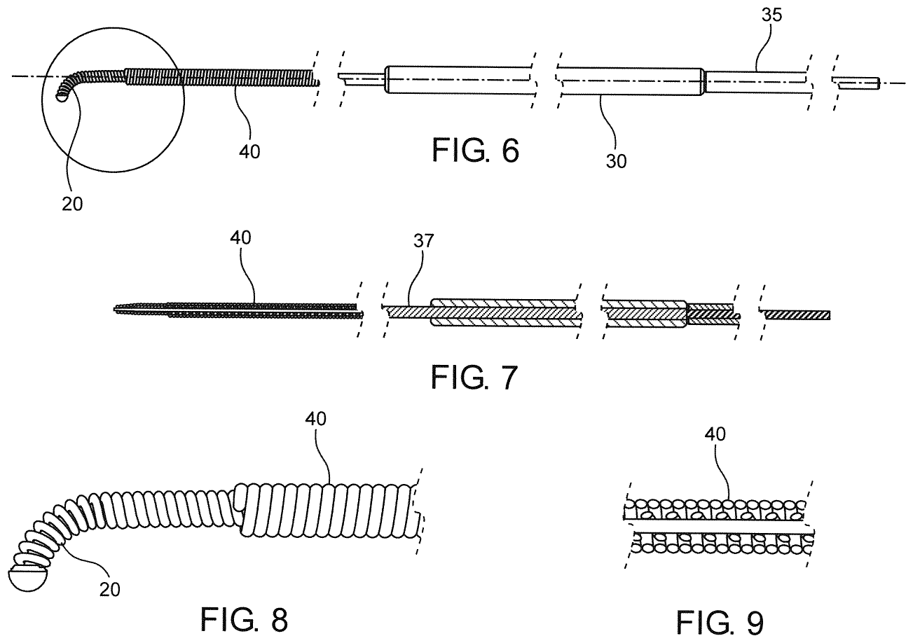

As shown in FIGS. 1-12, especially FIGS. 1-3 and FIGS. 6-9, a medical guide wire 10 for improved blood vessel navigation may comprise a wire. In one non-limited embodiment the wire is a substantially straight wire 10a (other than portions specifically described as not substantially straight). Guide wire 10 may have a curved distal tip 20. In some cases the distal tip 20 is curved in a manner that makes it j-shaped, for example as is shown in FIG. 1 and in FIG. 2. In some embodiments, there can be other curved shapes of the distal tip 20. As shown in FIG. 8, the radius of the distal tip may be about 3 millimeters. However, depending upon the intended location or other factors, the radius may vary. Furthermore, the shore hardness and/or Young's modulus of the distal tip 20 may vary as well.

Guide wire 10 may also include a shaft 30 that covers at least part of the wire 10.

A coil 40 may affixedly cover a portion of the wire 10 that is proximal to the distal tip 20 and that is distal to the shaft 30. In some embodiments, the coil 40 is made of a metal alloy of nickel and titanium, such as Nitinol. The work element 40 (coil 40) and the proximal portion of the whole guide wire may be somewhat less rigid than other parts of the guide wire 10 and hence may be a little less straight but in some embodiments are still substantially straight.

As seen from FIG. 1 and FIG. 2, the shaft 30 may be configured to be moved linearly toward the coil 40 so as to compress the coil. This compression may induce a moment that may rotate the distal tip 20.

In some cases, a full compression may induce up to 720 rotational degrees of rotation of the distal tip 30. In other implementation, the amount of induced rotational degrees may be 30-60, 60-90, 90-120, 120-150, 150-180, 180-210, 210-240, 240-270, 270-300, 300-330, 330 to 360, 390-420, 420-450, 450-480, 480-510, 510-540, 540-570, 570-600, 600-630, 630-660, 660-690 or 690-720 rotational degrees or any combination of minimum and maximum amounts in any of these ranges (for example up 30 to 720 degrees or 90 to 600 or up to 90-270 degrees or up to 120 to 630 or 30 to 90).

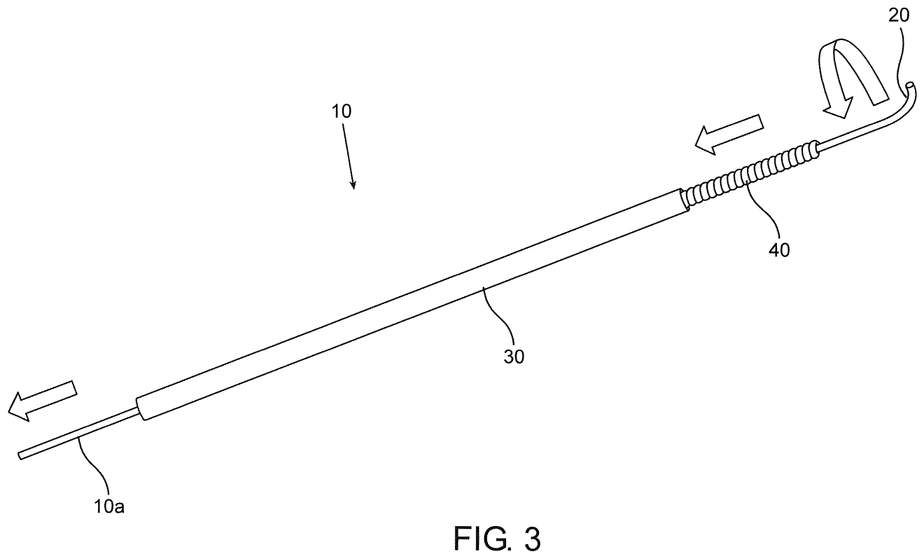

As shown in FIG. 3, reversing the linear movement of the shaft 30, in some implementations by letting go of the shaft 30 after it was moved distally toward the coil 40, releases the compressive force on the coil 40 and results in rotating the distal tip 20 in an opposite direction by any of the amounts above listed for the initial rotation.

In some embodiments, the distal tip 20 is radiopaque. For example, the distal tip 20 may be made of a platinum-iridium alloy that allows clear visibility of the distal tip 20 in an image of an X-ray, a CT, an MRI or another imaging technology.

In some embodiments, guide wire 10 further comprises a tube 35, for example a proximal tube 35, inside the shaft 30 along all or part of the length of the shaft 30.

The guide wire 10 may be constructed in a novel manner for example using bonded UV glue or welding laser to affix the coil 40 (or working element 40) on for example the distal tube 37 (which may be inward of the coil 40) such that coil 40 can still compress and expand when the shaft 30 presses coil 40. This is only implementation. In one further non-limiting implementation, the glue or welding laser connection between the coil 40 and the distal tube 37 may be located on the ends of the coil 40 (work element 40) such that the area between the two ends of the coil 40 can change in length due to the compression.

In some embodiments, the guide wire 10 is in combination with a microcatheter that is configured to be inserted into the blood vessel after the guide wire 10 has been inserted at the desired location.

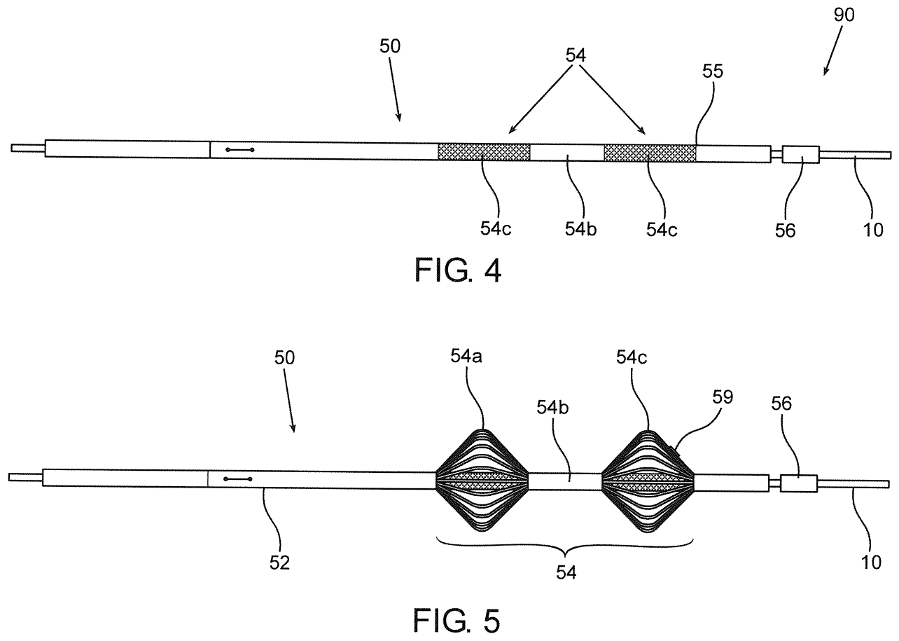

Another embodiment is a combination 90 (FIG. 4) of a microcatheter and a guide wire. Regarding the microcatheter, for example, as shown in FIG. 3 and FIG. 4, the microcatheter 50 may comprise a tube 52 having a braided distal portion 54 that is foldable axially.

The guide wire of the combination 90 may be any embodiment of guide wire 10 or it may be an entirely different guide wire or it may be a guide wire that contains a mechanism for triggering an axial folding of the braids of the braided microcatheter. Similarly, if the guide wire is an embodiment of guide wire 10, then the combination 90 may comprise guide wire 10 in combination with any microcatheter (even one that is not braided).

The guide wire of the combination 90 may be situated inside the microcatheter (after the microcatheter is inserted into the blood vessel). The guide wire may comprise a wire, for example a substantially straight wire, having at its distal end a mechanism configured to actuate an axial folding of the braided distal portion 54 of the microcatheter 50.

In some embodiments, the outer diameter of the microcatheter (before any axial folding) is between about 0.9 mm and about 1.2 mm, or 2 to 3 French size. The outer diameter of the braided portion 54 after axial folding, in one non-limiting embodiment, varies from a minimum outer diameter of about 2.0 mm to a maximum outer diameter of about 5.0 mm. However, in other embodiments, the outer diameter can be more or less than those numbers since the outer diameter needed depends on the size and location of the blood clot to be reached (and removed, for example by catching it) and on other factors.

As shown in FIG. 4, where the microcatheter is the braided microcatheter 50, the braided distal portion 54 of the microcatheter 50, in a neutral or an unfolded position, has a first outer diameter, a maximum first outer diameter or an average first outer diameter. In a folded position, as shown in FIG. 5, after axial pressure has been applied by the mechanism 56—for example the mechanism 56 on guide wire 10, the braided distal portion 54 of microcatheter 50 has a second outer diameter such that the second outer diameter is larger than the first outer diameter (or such that a maximum second outer diameter or an average second outer diameter is larger than a maximum first outer diameter or an average first outer diameter).

Within a particular braided section, for example braided section 54a, 54c of braided portion 54 (or within the overall distal braided portion 54) shown in FIG. 4, the outer diameter of the braided section (for example the outer diameter of the widest portion of the folds) may vary. Accordingly, for example, the braided distal portion in an unfolded position may have a maximum first outer diameter of, X, and in a folded position may have a maximum second outer diameter, Y, such that the maximum second outer diameter, Y, is larger than the maximum first outer diameter, X. For example, the maximum second outer diameter may be at least twice the maximum first outer diameter (or another multiple).

In another example, the braided distal portion 54 in an unfolded state (position) has an average first outer diameter and in a folded state (position) has a maximum second outer diameter such that the maximum second outer diameter is at least twice the average first outer diameter.

The word “twice” is just an example. The increase in outer diameter or in maximum outer diameter may be other multiples such as about 1.3, 1.5, 1.7, 1.7, 2.1, 2.3, 2.5, 2.7, 2.9, about 3.0 or anything in between these multiples.

In one implementation, the mechanism for actuating the axial folding comprises a mechanical actuator on the guide wire, which guide wire may be a guide wire 10 with features for rotating the distal tip or another guide wire without such features. One non-limiting example of such a mechanical actuator (which may be situated on guide wire 10 or another guide wire) is a bump 56 or a bulge 56 or a flange 56 such that pulling the guide wire proximally induces axial pressure against a distal end 55 of the braided portion 54 or against a distal end of the microcatheter 50 or against another portion distal to the braided portion 54 (such that this would put pressure on the braided portions indirectly). For example a flange 56, bump 56 or annular bulge 56 on guide wire 10 may press against a distal end 55 of a braided distal portion 54 of the microcatheter or a distal end of the microcatheter 50 or against a portion distal to the braided portion 54.

Another value of the axially folded strands forming braided portion 54 is that the strands of the braid—when they are folded and have an increased outer diameter—will serve as an embolic protection system that prevents an embolus such as a blood clot or air bubble from travelling to the brain during the procedure.

In the combination 90 that includes a braided microcatheter 50, the braided distal portion 54 may comprise a mixture of thicker strands and thinner strands, for example a symmetrical mixture, of thicker and thinner strands. The strands themselves may be made of Nitinol, a metal alloy of nickel and titanium. In one non-limiting implementation, the thicker strands are about 100 microns and the thinner strands are about 50 microns. In another non-limiting implementation, the outer diameter of the strands may be entirely different.

The shore hardness and/or Young's modulus of the microcatheter may vary. In addition, the shore hardness and/or Young's modulus of the braider distal portion 54 may also vary. The braided distal portion 54 may also vary in length. In one non-limiting embodiment, the length of the braided distal portion 54 is about 10-25 millimeters or anything in between (depending on the expected size and location of the blood clot, for example, among other factors). In another embodiment, its length is about 15 to 20 millimeters. In one non-limiting embodiment, the length of the braided portion 54 is between 16 mm and 19 mm or between 17 mm and 18 mm.

In some embodiments, the strands include radiopaque strands. For example, the strands may be made of a platinum-iridium alloy that allows clear visibility of the distal tip 20 in x-rays and other imaging technologies. Alternatively, or in addition, there may be one or more radiopaque markers 59 on the strands, as shown in FIG. 5.

In one example, as seen in FIG. 4, the braided distal portion comprise a first braided portion 54a and a second braided portion 54c, for example separated by a non-braided portion 54b. In another example, there may also be three braided portions or sections separated from one another by two non-braided sections so as to present five alternating braided and non-braided sections (not shown). Other implementations may include more than five braided and non-braided sections, for example alternating braided and non-braided sections. Furthermore, the lengths or relative lengths of the braided and non-braided sections are not necessarily as shown in the example of FIG. 4. In addition, in certain embodiments, one braided section (for example braided section 54a) may have a greater or lower outer diameter or maximum outer diameter or average outer diameter than another braided section (for example braided section 54c).

Optionally, in order to induce a controlled buckling or axial folding of the braided sections (for example of braided sections 54a, 54c) at particular points, particular junctures of the braided sections or braided portion—may be weaker, for example junctures between the braided and non-braided portions may be weaker. This is not a requirement.

As noted, the combination 90 (of guide wire and microcatheter) may include guide wire 10. For example, guide wire 10 may comprise a wire such as a substantially straight wire 10a that has a curved distal tip 20 (in any version described herein). The guide wire 10 may also include a shaft 30 covering at least part of the wire and a coil 40 affixedly covering a portion of the wire proximal to the distal tip 20 and distal to the shaft 30. In addition, moving the shaft 30 linearly toward the coil 40 may compress the coil 40 and rotate the distal tip 30. Releasing the shaft 30 for example by moving it back linearly or for example by just letting go of it in some embodiments may rotate the distal tip 20 in an opposite direction.

The braided portion 54, for example braided distal portion 54, of microcatheter 50 is configured to catch a blood clot alongside the braided portion 54—for example braided distal portion 54—when the microcatheter 50 is in the blood vessel. Accordingly, when braided distal portion 54 folds axially and the sections 54a, 54c expand outwardly/radially they create potential snagging points or snagging regions for any blood clot located alongside the microcatheter 50 in the blood vessel.

After the blood clot is caught in the strands of the braided section of the microcatheter 50, when the microcatheter 50 is withdrawn from the blood vessel, the blood clot is likewise removed automatically with the microcatheter.

In the case of an aneurism, the main value is the guidewire and its ability to navigate difficult to reach blood vessels where the aneurism is situated. Once the aneurism is reached using the guide wire 10, treatment of the aneurism is initiated. The treatment may be implemented in one of several ways, for example by endovascular coiling, covered stents or biological grafts.

Another option for the microcatheter 50 portion of the combination 90 or the microcatheter 50 alone, is fiber optics. The guide wire 10 may be removed and a fiber optic wire may in inserted at the desired location, such as the blood clot. Then in accordance with an algorithm, the clot composition and texture can be identified. For example, it can be determined whether the blood clot contains calcium or another specific compound.

Another embodiment is a microcatheter 50 (not in combination with a guide wire) comprising a tube having a braided portion 54, for example a distal braided portion 54 that is axially foldable. Microcatheter 50 may include any version described herein with respect to the combination 90. For example, the braided distal portion 54 may comprise a (symmetrical) mixture of thicker and thinner strands. For example, the braided distal portion 54 may comprise a first braided portion and a second braided portion separated by a non-braided portion (or more than two braided sections separated (or not separated) by non-braided section). The braided distal portion 54 is configured to catch a blood clot alongside the braided distal portion when the microcatheter is in a blood vessel.

The braided distal portion in an unfolded state (position) has a first outer diameter and in a folded state (position) has a second outer diameter such that the second outer diameter is larger than the first outer diameter. The braided distal portion 54 in an unfolded state (position) has a maximum first outer diameter and in a folded state (position) has a maximum second outer diameter such that the maximum second outer diameter is larger than the maximum first outer diameter. The maximum second outer diameter may be at least twice the maximum first outer diameter, or any other multiple such as about 1.3, 1.5, 1.7, 1.9, 2.1, 2.3, 2.5, 2.7, 2.9, about 3.1, etc. or anything in between. The braided distal portion 54 in an unfolded state (position) has an average first outer diameter and in a folded state (position) has a maximum second outer diameter such that the maximum second outer diameter is at least twice the average first outer diameter. The braided distal portion 54 may include or consist of a metal alloy of nickel and titanium.

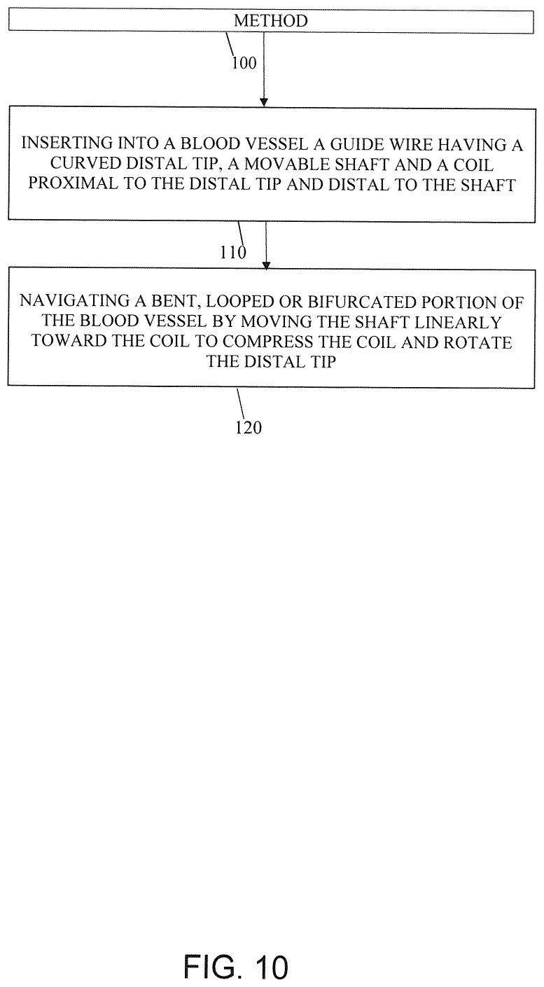

As seen in the flow chart of FIG. 10, another embodiment is a method 100 of inserting a guide wire into a blood vessel that contains a difficult-to-navigate portion. Method 100 may include a step 110 of inserting the guide wire into the blood vessel, wherein the guide wire includes a wire, for example a substantially straight wire, having a curved distal tip, a shaft covering part of the wire and a coil affixedly covering a portion of the wire proximal to the distal tip and distal to the shaft.

Method 100 may also include a step 120 of navigating a bent, looped or bifurcated portion of the blood vessel by moving the shaft linearly toward the coil so as to compress the coil, the compression rotating the distal tip (so as to more easily navigate the bent, looped or bifurcated portion of the blood vessel), so as to reach one of a blood clot, an aneurism and a hemorrhagic stroke.

Method 100 may also include a step of implementing a treatment for at least one of a blot clot, an aneurism (whether ruptured or unruptured) or a hemorrhagic stroke. For example, in the case of a blot clot, the treatment may involve inserting a microcatheter with an axially folding portion to the area of the blood clot and then expanding the diameter of the axially folding portion (such as a braided section) to catch the blood clot followed by removal of the microcatheter along with the blood clot.

In the case of an aneurism, the treatment may involve inserting a microcatheter and treating the aneurism using one of (i) endovascular coiling, (ii) a covered stent and (iii) a biological graft. In some cases, this may involve inserting the microcatheter to the area of the aneurism and pushing a coil through the microcatheter, for example to perform an endovascular coiling. The treatment may alternatively involve inserting the microcatheter to the area of the aneurism and introducing a covered stent or biological graft to treat the aneurism. The microcatheter may be any of the microcatheters described herein including any version of microcatheter 50 or another microcatheter.

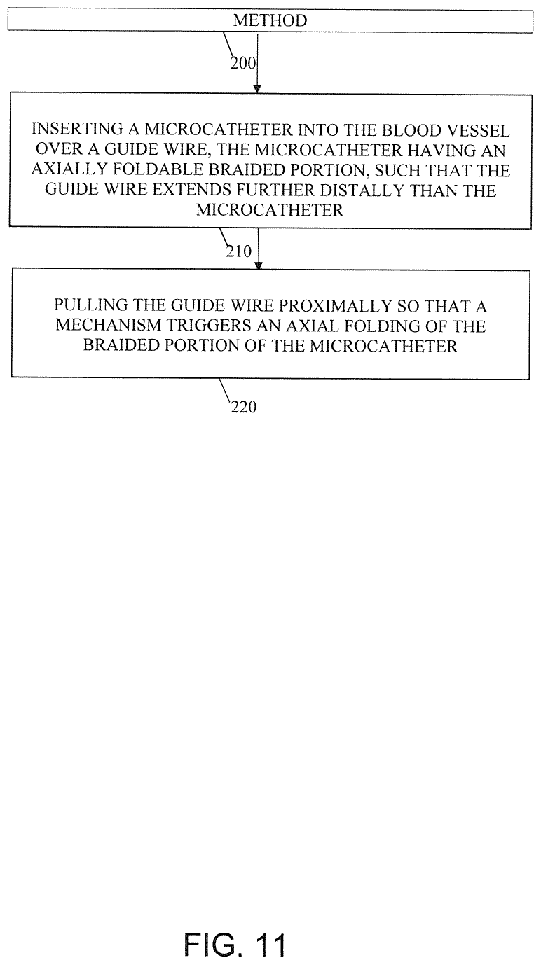

Another method, as seen in the flow chart of FIG. 11, may be a method 200 of removing a blood clot from a blood vessel of a mammalian subject. Method 200 may include a step 210 of inserting a microcatheter into the blood vessel over a guide wire. The microcatheter may have an axially foldable braided distal portion. The microcatheter may be extended over the guide wire such that a distal end of the guide wire extends further than a distal end of the microcatheter. In some versions there is a previous step of inserting the guide wire.

Method 200 may include a step 220 of pulling the guide wire proximally so that a mechanism 56 on the guide wire triggers an axial folding of the braided distal portion of the microcatheter 50.

Method 200 may further comprise axially folding the braided distal portion so as to catch a blood clot external to the microcatheter. Method 200 may also include a step of withdrawing the catheter with the blood clot.

In some implementations of method 200, the mechanism comprises a flange, bump or annular bulge that presses against a distal end of the braided distal portion (or against a portion distal to the braided distal portion) when the guide wire is moved proximally.

In some implementations of method 200, the guide wire is guide wire 10 such that in one example guide wire 10 comprises a wire, for example a substantially straight wire, having a curved distal tip, a shaft covering part of the wire and a coil affixedly covering a portion of the wire proximal to the distal tip and distal to the shaft such that linear movement of the shaft compresses the coil and rotates the distal tip.

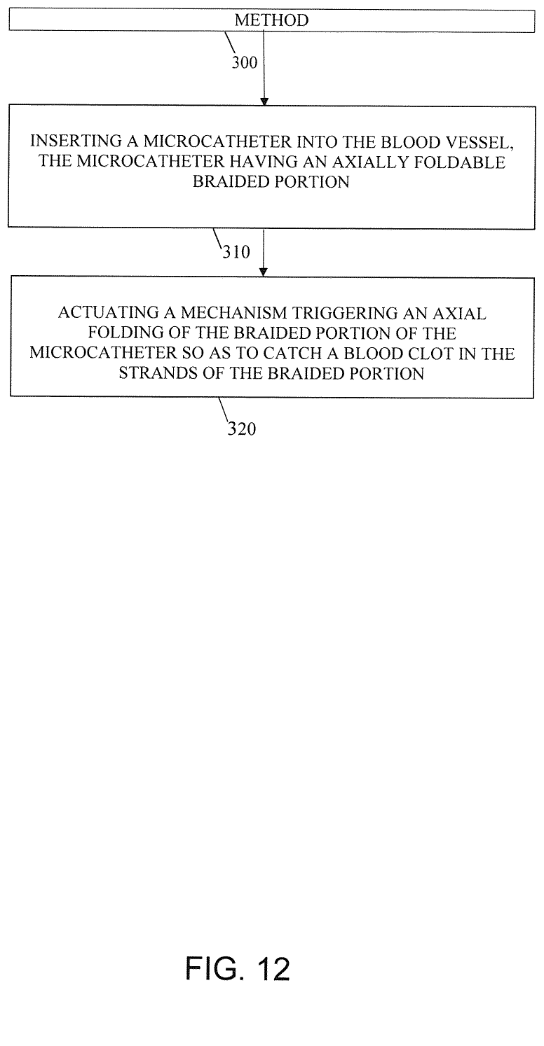

As shown in the flow chart of FIG. 12, a general method 300 includes a first step 310 of inserting a microcatheter into the blood vessel, the microcatheter having an axially foldable braided portion. Method 300 includes a further step of actuating a mechanism triggering an axial folding of the braided portion of the microcatheter so as to catch a blood clot in the strands of the braided portion. A further step may include removing the microcatheter along with the blood clot stuck to the braided portion of the microcatheter.

In any of methods 100, 200, 300, the microcatheter utilized may be any of the versions of the microcatheter described herein including any of the versions of microcatheter 50 with or without any of the version of guide wire 10 or another guide wire.

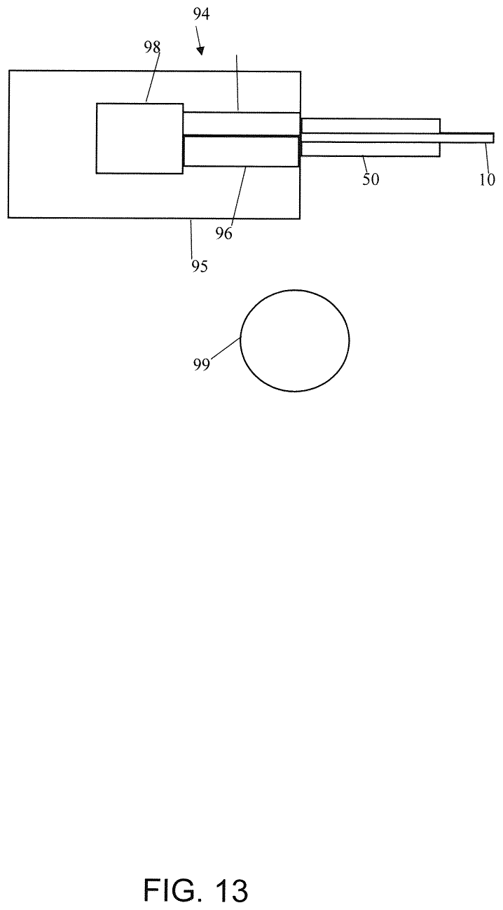

As shown in FIG. 13, another embodiment is a system 94 that includes a robot 95 and the combination 90 of a microcatheter and a guide wire. The robot 95 may for example include at least one motor. For example, it may include a first motor 96 that moves a guide wire such as the guide wire 10 and a second motor 97 that moves the microcatheter, for example microcatheter 50. In one implementation, an actuator 98 controlled by an input device 99 such as a joystick 99 actuates these movements. In system 94, the microcatheter utilized may be any of the versions of the microcatheter described herein including any of the versions of microcatheter 50 with or without any of the version of guide wire 10 or another guide wire.

While the invention has been described with respect to a limited number of embodiments, it will be appreciated that many variations, modifications and other applications of the invention may be made. Therefore, the claimed invention, as recited in the claims that follow, is not limited to the embodiments described herein.

1-40. (canceled)

41. A medical guide wire for improved blood vessel navigation, comprising:
a wire having a curved distal tip;
a shaft covering at last part of the wire;
a coil affixedly covering a portion of the wire proximal to the distal tip and distal to the shaft,
wherein moving the shaft linearly toward the coil compresses the coil and rotates the distal tip

42. The guidewire of claim 41, wherein moving the shaft linearly away from the coil releases the coil and rotates the distal tip in an opposite direction.

43. The guide wire of claim 41, wherein compressing the coil rotates the distal tip up to 720 rotational degrees.

44. The guide wire of claim 41, wherein compressing the coil rotates the distal tip from 90 to 720 rotational degrees.

45. The guide wire of claim 41, wherein the distal tip is radiopaque.

46. The guide wire of claim 41, wherein a radius of the distal tip is about 3 mm.

47. The guide wire of claim 41, further comprising a tube inside the shaft.

48. The guide wire of claim 41, wherein the coil is made of a metal alloy of nickel and titanium.

49. The guide wire of claim 41, in combination with a microcatheter.

50. The guide wire of claim 41, wherein the wire is substantially straight other than the curved distal tip.

51. A microcatheter and guide wire combination, comprising:
the microcatheter comprising a tube having a braided distal portion that is foldable axially;
the guide wire inside the microcatheter and comprising a having at a distal end a mechanism configured to actuate an axial folding of the braided distal portion of the microcatheter.

52. The combination of claim 51, wherein the braided distal portion in an unfolded position has a first outer diameter and in a folded position has a second outer diameter such that the second outer diameter is larger than the first outer diameter.

53. The combination of claim 51, wherein the braided distal portion in an unfolded position has a maximum first outer diameter and in a folded position has a maximum second outer diameter such that the maximum second outer diameter is larger than the maximum first outer diameter.

54. The combination of claim 51, wherein the braided distal portion in an unfolded position has an average first outer diameter and in a folded position has a maximum second outer diameter such that the maximum second outer diameter is at least twice the average first outer diameter.

55. The combination of claim 51, wherein the mechanism comprises a flange, bump or annular bulge such that pulling the guide wire proximally induces the flange, bump or annular bulge to press against a distal end of (i) the braided distal portion or (ii) a portion distal to the braided distal portion.

56. The combination of claim 51, wherein the braided distal portion comprises a mixture of thicker and thinner strands.

57. The combination of claim 51, wherein the braided distal portion comprises a symmetrical mixture of thicker and thinner strands.

58. The combination of claim 51, wherein the braided distal portion comprises a first braided portion and a second braided portion separated by a non-braided portion.

59. The combination of claim 51, wherein the wire has a curved distal tip, the guide wire also including a shaft covering at least part of the wire and a coil affixedly covering a portion of the wire proximal to the distal tip and distal to the shaft, wherein moving the shaft linearly toward the coil compresses the coil and rotates the distal tip.
